(19) United States
(12) Patent Application Publication
(10) Pub. No.: US 2006/0129211 A1
(43) Pub. Date: Jun. 15, 2006

(54) Method, apparatus and protocols for performing low level laser therapy

(76) Inventors: Luigi P. Canitano, Thornhill (CA); Don Fitz-Ritson, Toronto (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(21) Appl. No.: 11/271,768

(22) Filed: Nov. 14, 2005

Related U.S. Application Data
(60) Provisional application No. 60/626,866, filed on Nov. 12, 2004.

(51) Int. Cl. A61N 5/06

(52) U.S. Cl. 607/89

(57) ABSTRACT

Apparatus for delivering low level laser therapy comprising at least one probe for delivering the low level laser therapy; a processor, connected to the at least one probe, for transmitting signals to the probe corresponding to the low level laser therapy; wherein, after receiving the signals, the low level laser therapy is delivered by the probe at a wavelength of between 600 and 1100 nm.

### CROSS REFERENCE TO RELATED APPLICATIONS

This application claims the benefit of U.S. Provisional Application No. 60/626,866, filed Nov. 12, 2004, which is incorporated herein by reference.

### FIELD OF THE INVENTION

The invention relates generally to the treatment of various musculo-skeletal and soft tissue injuries, and, more particularly to a method, apparatus and protocols for performing low level laser therapy (LLLT).

### BACKGROUND OF THE INVENTION

Various studies have found low level laser therapy (LLLT) effective in providing relief of acute and chronic pain, increasing the speed, quality and tensile strength of tissue repair, stimulating the immune system, nerve function and cell regeneration, increasing metabolic activity, developing collagen and muscle tissue, reducing inflammation and promoting faster healing and clot formation. LLLT is non-invasive and delivers palliative and curative therapy for both humans and animals in a cost-effective, painless and drug-free manner.

LLLT uses light amplification by stimulated emission of radiation (“laser”) energy in the form of coherent and monochromatic light. This energy is supplied to the body in the form of non-thermal photons, administered at a predetermined wavelength, which varies from visible to non-visible collimated laser spectrums. When LLLT is applied to an area, the photon energy penetrates the skin, enters the tissues and is then incorporated by certain cell mechanisms for the synthesis of ATP (adenosine triphosphate). Stimulated emission through the cascading effect of photon energy is the basic mechanism often referred to as “biostimulation”. Biomedically, when LLLT is applied to injuries or wounds, the relief of acute and chronic pain conditions is achieved, inflammation is eliminated and faster, more effective healing of the tissue results

A variety of laser devices and therapeutic treatments have been proposed and are currently in use such as described in U.S. Pat. No. 5,464,436 to Smith entitled Method of Performing Laser Therapy, U.S. Pat. No. 6,663,108 to Salansky entitled Method and Apparatus for Localized Low Energy Photon Therapy (LEPT) and U.S. Pat. No. 4,413,267 to Dumoulin-White et al. entitled Therapeutic Laser Device and Method including Non-invasive Subsurface Monitoring and Controlling Means. U.S. Pat. No. 4,836,203 to Muller; Gerhard (Berlin, Del.); Greve; Peter (Essingen, Del.) describes a Device for Therapeutical Irradiation of Organic Tissue by Laser Radiation and U.S. Pat. No. 4,724,835 to Liss, Saul; Liss, Bernard S.; Krakower, Sam and Feygin, Ilya describes a Laser Therapeutic Device while U.S. Pat. No. 4,930,505 is directed at a Method of Enhancing the Well-Being of a Living Creature and U.S. Pat. No. 5,231,984 is directed at a Laser Therapeutic Apparatus.

However, many of the prior art laser therapies and devices for musculo-skeletal conditions do not promote full healing of the injury to full heal causing patients to experience further pain and the possibility of aggravating the injury.

Also, most current laser therapy devices are not portable requiring patients suffering from acute or chronic musulo-skeletal conditions to visit their doctor, therapist or clinician in order to receive treatment. This may be time consuming and may cause further discomfort and the possibility of aggravating the condition. Lack of portability of these other devices results in certain evident limitations when treating within the sports field. The lack of portability means that injuries that occur in the field cannot receive required immediate treatment. This affects the recovery process as studies suggest that immediate treatment to an injury provides quicker recoveries.

It is, therefore, desirable to provide a method and apparatus which overcomes some of the disadvantages of the prior art.

### SUMMARY OF THE INVENTION

It is an object of the present invention to obviate or mitigate at least one disadvantage of previous laser therapy devices.

The invention provides a method, apparatus and protocols for treatment of a musculo-skeletal condition or injury by delivering low level laser energy at a pre-determined and pre-selected frequency, cadence, duty cycle and wavelength to provide an optimum biostimulation of tissue with maximum penetration for a precise dosage period.

The method of treatment includes the provision of a diagnosis of the condition or injury, selection of pre-determined treatment settings for the diagnosed disorder, delivery of laser energy to the afflicted area for a predetermined treatment time, monitoring of the treated area upon completion of the treatment cycle and repeating the steps of diagnosis and delivery based upon the results of the monitoring step.

Another aspect of the invention is an apparatus designed to effectively administer the treatment protocols, the laser device comprising a control device having a software operating system (microprocessor), a single probe head with microprocessor and display, a cluster probe head with microprocessor and display, an AC/DC power supply adapter interfaced with a conventional plug, an alternate battery power source, a keyed locking element, removable safety plug and emergency stop button, a microprocessor to access the pre-determined treatment protocols and pre-programmed treatment protocols.

The invention involves the administration of the laser light from the apparatus for a predetermined time interval using a pre-selected setting for cadence, frequency, duty cycle and energy output. Each of these factors, in proper combination, promotes the efficient healing of the treated tissue and the abatement of the pain associated with the various disorders.

The apparatus delivers laser photon energy in pre-determined dosages under predetermined frequency, cadence, duty cycle and wavelength for a pre-determined dosage period to the afflicted tissue. The frequency determines the amount of Joule energy delivered to the injured tissue area while the duty cycle modifies the frequency output working in tandem with the cadence.

In another aspect, there is provided apparatus for delivering low level laser therapy comprising at least one probe for delivering the low level laser therapy; a processor, connected to the at least one probe, for transmitting signals to the probe corresponding to the low level laser therapy; wherein, after receiving the signals, the low level laser therapy is delivered by the probe at a wavelength of between 600 and 1100 nm.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 is a schematic diagram of apparatus for controlling and administering treatment for pain and soft tissue injuries.

FIG. 2 is a broken away view of a probe for use with the apparatus of FIG. 1.

FIG. 3 is a schematic view of a second probe for use with the apparatus of FIG. 1.

FIG. 4 is a schematic view of the housing of FIG. 1.

FIGS. 5 and 6 are flowcharts outlining a method of using the apparatus of FIG. 1.

FIG. 7 is a flowchart outlining a second method of treating a musculo-skeletal condition.

FIGS. 8 to 15 are sample treatment protocols for treating a musculo-skeletal condition.

### DETAILED DESCRIPTION

Generally, the present invention provides a method, apparatus and protocols controlling and administering low level laser therapy treatment for a musculo-skeletal condition or injury.

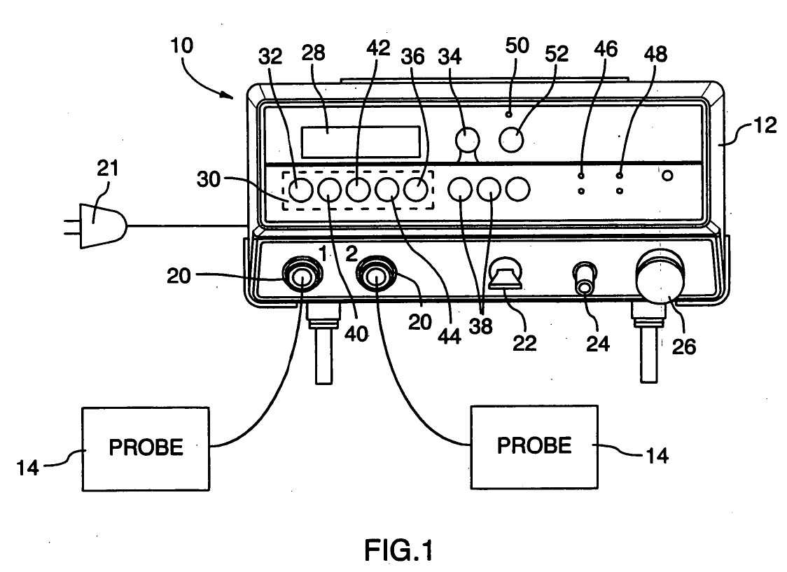

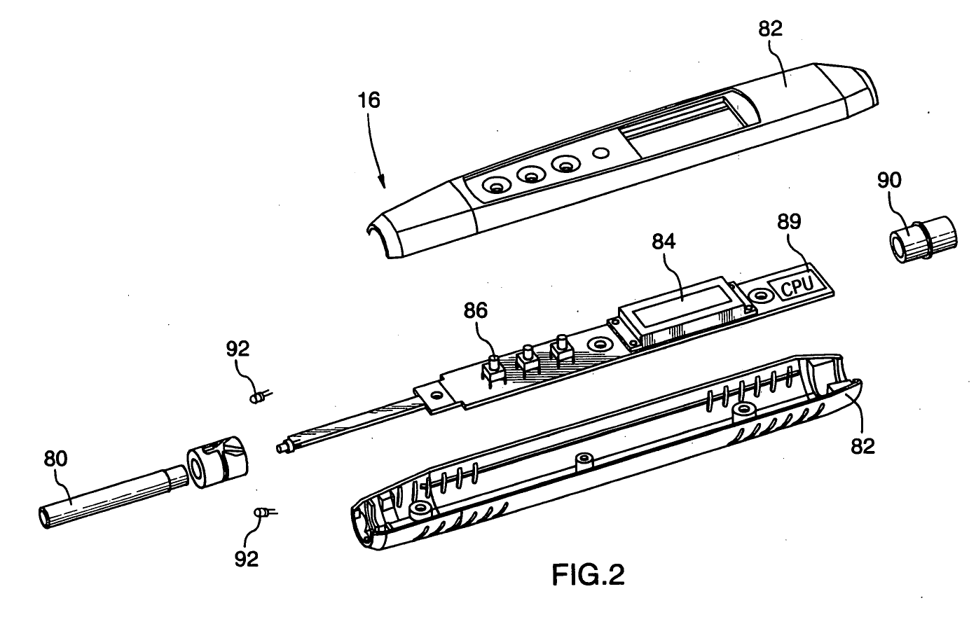

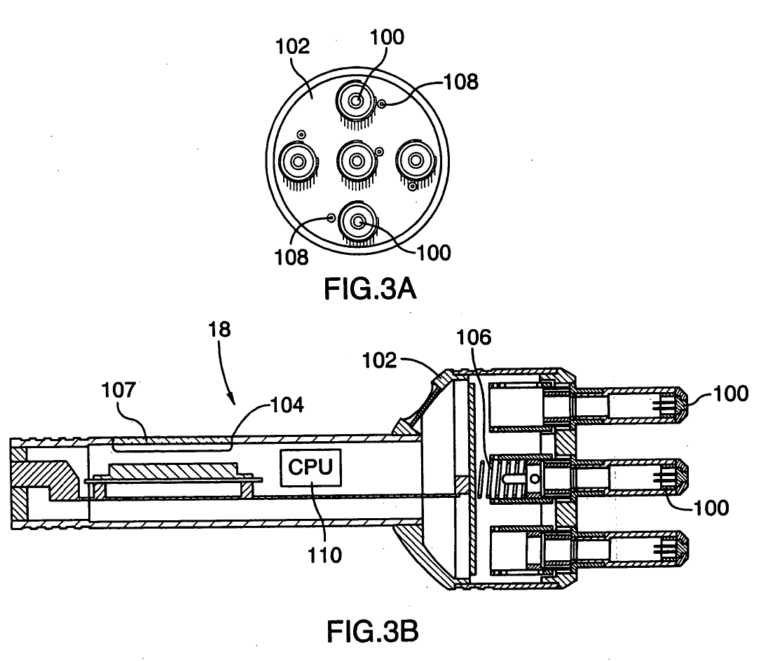

Turning to FIG. 1, a front view of apparatus for controlling and administering low level laser therapy treatment is shown. The apparatus 10 comprises a control device 12 coupled to at least one probe 14 for administering the low level laser therapy treatment. The probe 14 may be a single probe 16 such as shown in an exploded view in FIG. 2 or a cluster probe 18 as schematically shown in FIGS. 3a and 3b. Each probe 14 is connected to the control device 12 via individual probe connectors 20. Each of the probe connectors 20 represents a separate channel over which the connected probe 14 operates to deliver the low level laser treatment to the patient. In the present embodiment, there are two operating channels, identified by the 1 and 2 above the probe connectors 20. When the device is in use, only one of the channels will be operating at a time such that only one of the probes is administering the treatment.

The control device 12 is preferably powered by a power source (not shown) connected to the end of a conventional plug 21. Alternatively, a battery, preferably rechargeable, may power the control device 12.

The control device 12 includes a keyed locking element 22 which controls the ON/OFF functionality of the device. The keyed element 22 allows operation of the control device 12 to be limited only to the clinician who is in possession of the associated key. Other safeguards such as a removable safety plug 24 and an emergency-stop button 26 are also provided to quickly power down the control device 12 when necessary. Operation of these further safeguards will be known to one skilled in the art.

The control device 12 also includes a display window 28 on which is displayed information concerning the current treatment protocol being administered to a patient among other information. The display window 28 allows a clinician or the patient to observe and track parameters, such as the remaining treatment time, of the treatment protocol and serves as an information interface between the control device 12 and the clinician and/or patient (“user”).

The control device 12 further comprises a set of treatment control buttons 30 which include, but are not limited to, a program (P) button 32, a treatment protocol (TP) button 34, time (T) button 36, a pair of value buttons 38, a cadence (CD) button 40, a Duty Cycle (%) button 42 and an Energy (E) button 44.

A first set of, preferably two, LED display lamps 46 provide identification to the clinician which channel (1 or 2) is in use, and subsequently which probe 14 is in use. If the LED display lamp 46 adjacent the number 1 is illuminated, this indicates that the probe 14 connected to the channel 1 probe connector 20 is in use. Similarly, if the LED display lamp 46 adjacent the number 2 is illuminated, this indicates that the probe 14 connected to the channel 2 probe connector 20 is in use. Each probe connector 20 is capable of receiving the single probe 16 or the cluster probe 18 and includes sensors (not shown) to determine which probe has been connected. The sensors receive signals from the probe indicating information such as the number of diodes and the diode characteristics. The sensors then transmit this information to a CPU 60 in the control device 12.

A second set of LED display lamps 48 preferably displays information related to the insertion of the plug 21 in a power supply to provide power to the control device and to charge the battery when it is in a low condition. A third set of LED display lamps 50, associated with a standby/ready button 52, indicates to the user when the control device 12 is in a stand-by mode. In the stand-by mode, information, such as the default settings for each connected probe 14, or the selected probe and a prompt for treatment protocol selection, is displayed in the display window 28.

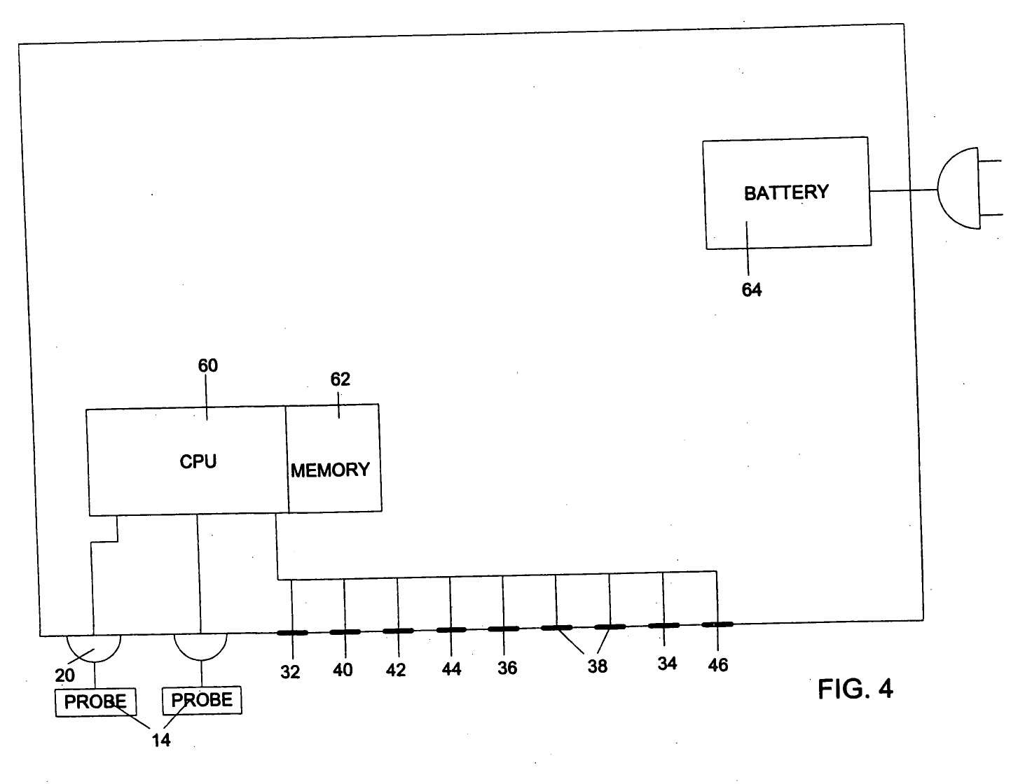

Within the control device 12, as shown in FIG. 4, is the CPU (seen as a microprocessor) 60 connected, and in communication with, a memory, or memory processor, 62 which stores a plurality of pre-determined treatments, in the form of laser treatment protocols, representing information for the delivery and regulation of an optimal amount of photon energy through regulation of cadence, frequency and duty cycle which result in the output energy of the lasers of the probes. The memory 62 of the CPU 60 also stores a plurality of settings to deliver laser energy at a preferred wavelength of 905 nm in a predetermined/pre-selected dosage.

If the probes have display windows, this output energy may be displayed on the probes. In this embodiment, the control device 12 also houses the, preferably rechargeable, battery 64. The rechargeable battery is preferably connected to a charging mechanism so that the battery is charged when the plug 21 is connected to a power supply. It will be understood that there are other parts which are located within the control device but are not presently shown as they are not critical to the implementation of the current invention.

The CPU 60 is internally connected to the probe connectors 20 to transmit and receive signals from the connectors 20 and their connected probes 14. The CPU 60 is also connected for communication with the set of treatment buttons 30, the locking element 22, the removable safety plug 24 and the emergency-stop button 26 and the memory 62.

In operation, when the control device 12 is initially started up (turned on) via the keyed locking element 22, default pre-settings cycles are displayed in the display window 28. In the preferred embodiment, the default pre-settings cycle display such information as the condition of the battery, a name of the manufacturer, a name of the control device, the last update of the software operation system, information concerning the attached probes and a request for protocol selection. It will be understood that this is simply an example of the type of information which may be displayed in the pre-settings cycle.

Pressing the P button 32 allows the user to select the desired channel (or probe) for delivery of the low level laser therapy. As discussed above, the probe connectors 20 include a sensor to determine whether a single probe or a cluster probe is connected and transmits a signal, containing this information, to the CPU 60 so that the CPU displays the correct pre-settings on the display window 28 in accordance with the selected channel and associated probe type. Once the selected channel is determined in conjunction with the desired probe 14, the TP treatment protocol button 34 is depressed to display a first treatment protocol in the display window. When the TP button 34 is pressed, a signal requesting this information is transmitted to the CPU 60 which accesses the memory 62 to retrieve this information. After retrieving the information, the CPU displays it on the display window 28. In the preferred embodiment, the parameters of the treatment protocols may not be varied or modified in any way as the pre-set treatments are preferably designed in accordance with clinical research. Therefore, re-adjustment of the Frequency, Cadence, Duty Cycle and Time is not possible in the preferred embodiment.

Depressing the value keys 38 allows the user to scroll through all of the pre-stored treatment protocols to select the treatment protocol corresponding to the type of injury to be treated. As discussed above, the treatment protocols are predetermined and each comprises a plurality of low level laser treatments.

After the treatment protocol has been selected, the CPU 60 retrieves the information concerning the selected treatment protocol from memory 62 and controls the delivery of the selected treatment protocol, in the form of low level laser therapy, to the laser diodes in the probe 14 as will be discussed below.

In an alternative embodiment, the pre-programmed settings for each of the treatment protocols such as cadence, frequency, duty cycle, output energy and treatment time to permit a pre-selected dosage of laser energy (in Joules/cm²) to be emitted from the probe 14 are variable. This may be achieved by including a Maintenance/Service mode during the Treatment Protocol selection stage, whereby the user may independently select the desired Frequency, Cadence, Duty Cycle and Time. The elements of the selected dosage can be varied by use of the appropriate element button and the value set of value buttons 38. This feature is described in more detail below.

If the user wishes to change the parameters of the frequency, cadence, duty cycle and time of the treatment protocol, the user may enter into the Maintenance/Service mode. In order to change the frequency (in Hz) of the treatment protocol within the Maintenance/Service protocol, the clinician presses the P button 32 to view the current frequency. The frequency may be changed via the set of value buttons 38 from 30 to 10000 Hz. Furthermore, the cadence can be displayed by pressing the CD button 40 and also changed with the value buttons 38 allowing selection of a cadence setting in a range from 2 to 28 Hz. However, cadence selections must be made in combination with the selection of a duty cycle since the duty cycle determines the time in percentages on which the pre-determined pulse widths are cycling on and off. The duty cycle may be displayed by pressing the Duty Cycle (%) button 42. When the duty cycle selection is set at 50%, the pulse train operates in equal proportions, being on and off during one complete cycle of a specific pulse width, preferably 375 ns. The duty cycle is utilized in 10% incremental steps between 10% to 90% by using the set of value buttons 38. A value of 100% disables any pre-selected value of cadence and causes the apparatus 10 to be on throughout the entire treatment time at the pre-selected value of frequency. The amount of laser energy emitted from the selected probe 14 is displayed by pressing the E button 44 which will typically display a value from 0.01 to 288 Joules/cm². The treatment protocol therapy time is determined as a result of the amount of laser energy in joules/cm² delivered.

The control device 12 is preferably programmed with a therapy time of five minutes within each treatment protocol, however, other therapy time periods, such as two minutes, are contemplated. Pressing the T button 36 causes the therapy time to be displayed in the display window 28. In only the Maintenance/Service mode, the therapy time may be changed using the set of value buttons 38. The therapy time may be pre-selected from 60:00 (60 minutes) to 01:00 (1 minute) in one minute increments. Pre-setting of the therapy time allows the user to determine the time period for which the connected probe 14 delivers the treatment protocol via the pulse train resulting in the specified treatment protocol dosage to the injured area. This also allows the selected treatment protocol being delivered to the afflicted area or tissue to be regulated. Upon expiry of the therapy time, power to the probe 14 delivering the treatment is automatically shut off by the CPU 60.

Turning to FIG. 2, an exploded view of the single head probe 16 is shown. The single head probe 16 preferably uses a single fixed laser diode 80 mounted on an elongated pen shaped handle 82. The laser diode preferably provides low level laser therapy at a wavelength between 600 and 1100 nm, and more specifically in a range between 905 and 1100 nm and most specifically at a wavelength of 905 nm. The probe 16 preferably comprises a display window 84 for displaying information concerning the treatment protocol parameters of frequency, cadence, duty cycle, time and output energy being administered, along with a set of buttons 86 which provide the user with the capability of interacting with the control device 12 and a set of LEDs 88 which reflect the operational status of the probe 16. At an end opposite the laser diode 80, the probe 16 comprises a connector 90 for mating with the probe connector 20 of the control device 12. A CPU 89 is also preferably located within the probe 16. It will be understood that the probe may be a standalone unit with the CPU 89 performing the functions of the CPU 60 in the control device 12.

A light, preferably a green LED, on the laser diode 80 is turned on immediately after a “Stop/Start” button (part of the set of buttons 86) along with a set of pilot lights 92 which are activated prior to the activation of the laser beam. In a preferred embodiment, there are 2 red LED pilot lights 92 mounted in the head of the single probe 16 to indicate the direction of the laser beam and to illuminate the area being treated. The probe 16 also includes a Pause button (within the set of buttons 86) which allows the treatment to be paused and then continued from the same time interval so that the treatment time remains a constant time period. This is achieved by pressing the pause button once. In order to re-activate and continue the treatment protocol, the StarUStop button is pressed.

When the probe 16 is initially turned on, the CPU 60 performs several tests to confirm that the laser diode is operational and is in range of operable tolerances. Alternatively, the CPU 89 may perform this functionality. In the present invention, the CPU 60 includes a list of expected, or threshold values for the laser diode (such as the internal impedance of the laser diode) and if the threshold value is not met, the CPU 60 does not provide any power to the probe 16 for operation resulting in an error message being displayed. The CPU 60 then preferably defaults to re-perform the tests. If the probe 16 passes the start up testing, the probe 16 preferably displays a “Ready” message in the display window 84, and/or display window 28.

In one embodiment, the probe 16 preferably delivers laser photon energy in a range from 0.06 Joules/cm2 to 19.20 Joules/cm2, at a wavelength of 905 nm and a maximum power of 20 W of peak power and a pre-determined at a pulse width of 375 ns.

Turning to the cluster probe 18 of FIGS. 3a and 3b, the cluster probe 18 comprises five laser diodes 100, preferably each with a wavelength of 905 nm providing a power of 5×20 W and mounted in a circular head 102 which itself is mounted on a tubular handle portion 104. The laser diodes 100 preferably have pilot lamps 108 which indicate to the physician a direction of the laser beam. A display window 107 is also provided within the probe 18. Although not shown, the probe 18 may also include a set of treatment control buttons. The laser diodes 100 are spring-loaded, as indicated by spring 106, to provide an ergonomically comfortable device and to allow appropriate surface contact to the afflicted area during repeated and prolonged use of the probe 18 in treatment. The probe 18 also includes a CPU 110 for receiving and transmitting messages with the CPU 60 or for performing the functionality of the CPU 60 in order to provide a standalone, portable unit. In the preferred embodiment, the laser diodes 100 are spaced in a range of from 2 mm to 2.75 mm from each other to provide maximum coverage of laser energy to a proposed treatment area of up to 5.5 mm in diameter. It will be understood that this calculation is preferred for a cluster probe having five laser diodes and that other spacing is preferable for cluster probes having more or less laser diodes in the head 102. Each diode 100 preferably emits laser photon energy aperture of 200 μm (width)×10 μm (depth) with a beam divergence (FWHM) of 11°×25° in grad degrees. Mean average power at convergence is 0.1 mW to 60.0 mW per laser diode while the probe 18 preferably delivers laser energy from 0.32 Joules/cm² to 108 Joules/cm². The actual energy delivered is determined by the treatment protocol selected on the control device 10.

Along with the start-up testing, the CPU 60 also performs continuous testing to monitor the status of the diodes such that if one or more of the laser diodes becomes inoperable or is malfunctioning whereby it is not applying the predetermined amount of photon energy with an expected tolerance (preferably +/−5%), the probe 18 will display an error message and/or Not Complete and the control device 12 displays ERROR:(1) and subsequently will auto-turn off resulting in a default cycle. This extra step of testing provides further security in the operation of the apparatus 10. In the preferred embodiment, the diodes 100 are easily removable so that it is easy for a clinician to change a diode when it requires replacement.

If the cluster probe 18 fails the start up test, the control device 10 will then preferably re-start and re-boot (in order to re-execute the start up test), during which the CPU 60 attempts to re-detect any abnormality or an out of tolerance range for the diodes 100 which results in the control device 10 displaying the same error message. This continues until the CPU 60 detects that there is no abnormality and the diodes 100 (and associated circuitry) are operating within the accepted tolerance range at start up. This may require that one, or more of the laser diodes be replaced prior to use.

An internal monitoring system, preferably stored in the memory of the CPU 110 in the cluster head and 89 in the single probe, operates continuously whenever either the single or the cluster probe is administering treatment and delivering photon energy to the patient.

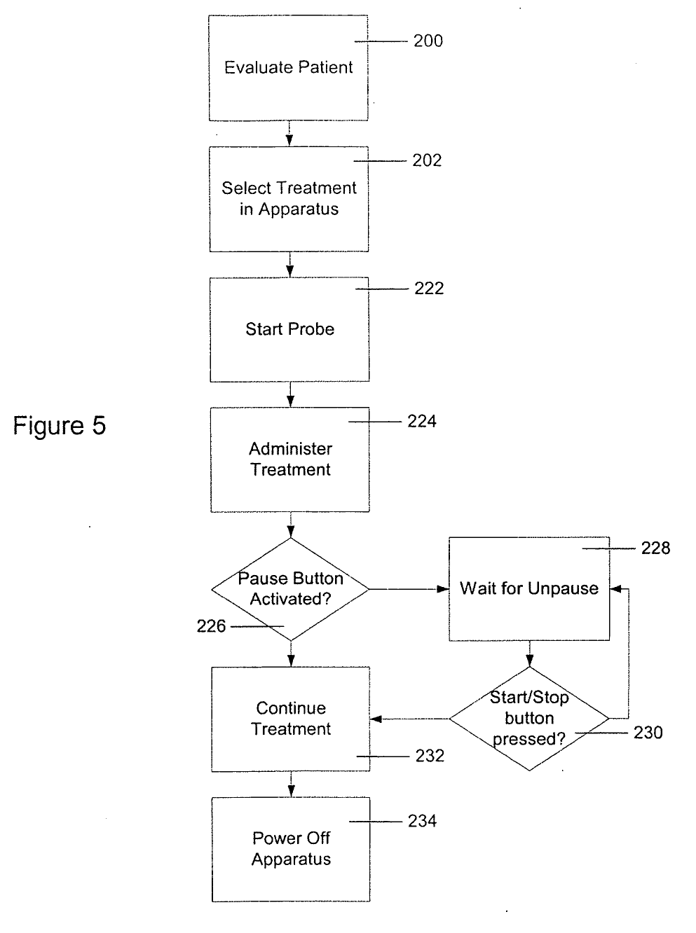

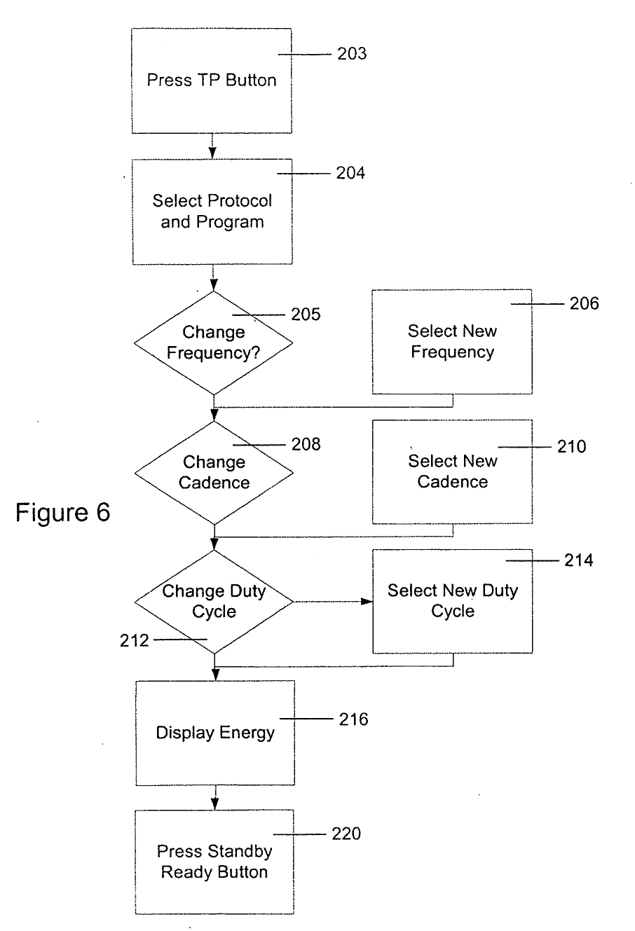

Turning to FIGS. 5 and 6, flowcharts outlining a first embodiment of using the apparatus of the invention for administering low level laser therapy in the form of a treatment protocol to an afflicted or injured area are shown.

As shown in FIG. 5, a patient is initially evaluated to determine the patient's injury which, in turn, allows the clinician to determine the type of treatment that the patient requires (step 200). After the type of injury has been determined, the physician then turns on the apparatus 10 and selects the associated treatment protocol, or the maintenance service mode, required for the patient (step 202). One embodiment of selecting the treatment protocols (step 204) is shown in FIG. 6.

In order to select the treatment, the TP button 34 is pressed (step 203) and a first of a list of treatment protocols is then displayed on the display window 28 from which the user may select a treatment protocol and program (step 204). This is achieved by using the set of value buttons 38 to scroll through the list of treatments and then pressing the P button 32 to select the treatment protocol/program. Each time one of the buttons 30 is pressed, corresponding signals are transmitted to the CPU 60 indicating the user's actions. The CPU 60 then processes the information and communicates with the memory 62 before transmitting signals to the display window, and/or the probes. As discussed above, one press of the P button 32 selects the probe connected to channel 1 and two presses select the probe connected to channel 2. If the user is in the maintenance/service mode, the user may decide to change the frequency of the treatment protocol (step 205). The new frequency may then be selected (in the manner as discussed above) (step 206). After the new frequency has been selected or if the user has decided not to change the frequency, the cadence of the treatment may be changed (step 208). The new cadence may then be selected (in the manner as discussed above) (step 210). After the new cadence has been selected or if the user has decided not to change the cadence, the duty cycle of the treatment may be changed (step 212). The new duty cycle may then be selected (in the manner as discussed above) (step 214). After the new duty cycle has been selected or if the user has decided not to change the duty cycle, the energy level of the treatment is displayed (step 216). Since the treatment is ready to be administered, the CPU 60 waits for the standby/ready button 52 to be pressed which indicates that the apparatus 10 is ready to administer the treatment (step 220). It will be understood that other parameters such as the length of the treatment may be changed as well.

After the treatment has been selected and set up (step 202), the probe 14 which has been selected for administering the treatment is started (step 222). After the start up testing is completed and passed (indicating that the probe 14 is in working order), the probe 14 is placed in light contact with the patient's skin and perpendicular to the area to be treated in order to apply the treatment (step 224). Simultaneously, the processor 60 transmits signals to the probe 14 corresponding to the selected treatment protocol to be applied by the diode(s) in the probe 14. In another embodiment, the laser therapy may be immediately applied after the CPU 60 provides the signals or the laser therapy may be controlled via buttons on the probe 14.

In this embodiment, after receiving the signals from the CPU 60, the CPU 110 or 89 in the probe determines the power level of the laser diodes and supplies the required voltage and current to the diodes for treatment. During the treatment, the CPU in the probe, or the CPU 60, constantly monitors the impedance level being experienced by the probe to monitor operation of the probe and to transmit the required instructions to the probe, such as power off, if the impedance levels are incorrect and out of range. Depending on the selected therapy time, the probe is held firmly for the predetermined time interval, which is preferably five minutes.

In most cases, the overall treatment program involves a stepwise treatment whereby the frequency, cadence, duty cycle and energy is altered within the steps of a specific treatment protocol as will be discussed below. Although it is preferred that each step of the stepwise treatment is of equal length, the length of time that the treatment remains at a certain level with respect to frequency, cadence, duty cycle and energy is determined by the individual who enters the treatment protocols into the memory 62 of the control device 12 or the probe 14 in the standalone unit embodiment.

During the treatment, the CPU 60 verifies whether or not a pause button has been pressed (step 226). The button is preferably located on the probe but may also be located on the control device. If the pause button is pressed, this indicates that the physician has decided to delay the administration of the treatment to the injured area. After the pause button press has been sensed, the CPU 60 waits for the treatment administration to be unpaused (step 228).

The processor continues to sense when the administration of the treatment is to be continued (step 230). When the processor senses that the treatment is unpaused, i.e. the Start/Stop button is depressed, the treatment is then continued (step 232) and the treatment cycle continues at the selected settings from the point in time when it was initially stopped.

After the treatment is completed, the apparatus may be turned off (step 234).

As a follow-up to the treatment, the afflicted or injured area is then preferably re-assessed and further treatment cycles are administered, if necessary.

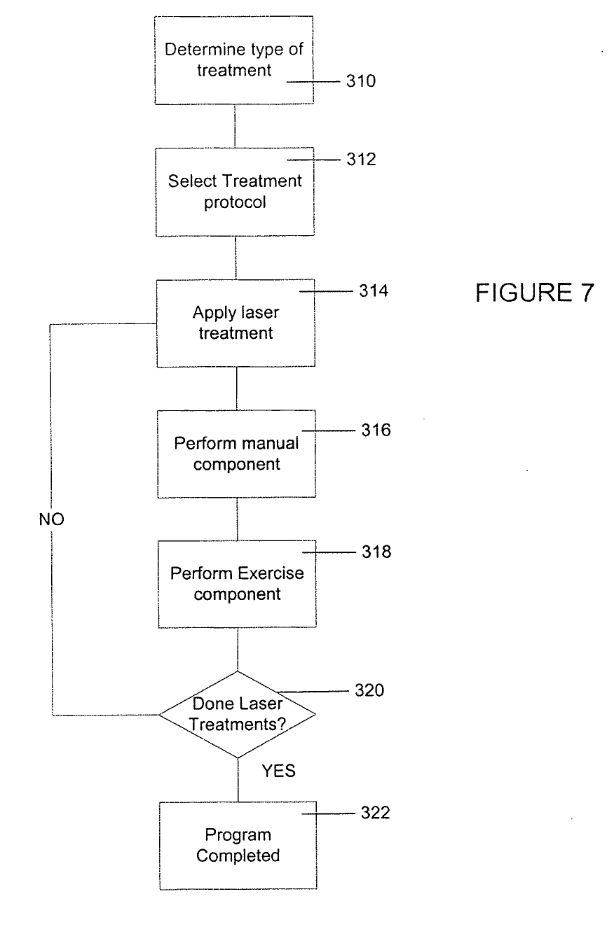

Turning to FIG. 7, a flow diagram illustrating another embodiment of treating musculo-skeletal injuries using the apparatus 10 is shown. In this treatment method, the method preferably comprises a laser component and an exercise rehabilitation component but it will be understood that the program may comprise the laser component with or without the exercise rehabilitation component. When an individual requires treatment for a musculo-skeletal injury, the first step is to examine the individual to determine the type of injury or musculo-skeletal condition. This allows a clinician to determine the type of treatment (step 310) which will assist in the recovery of the patient.

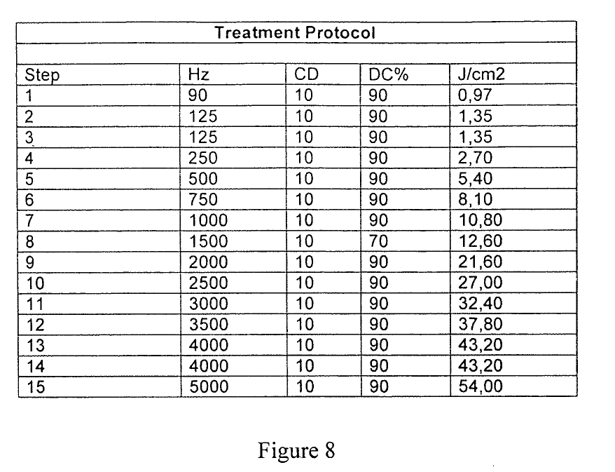

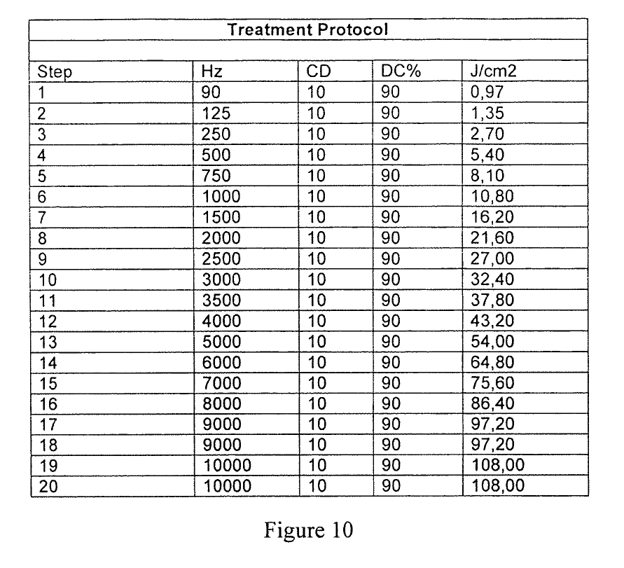

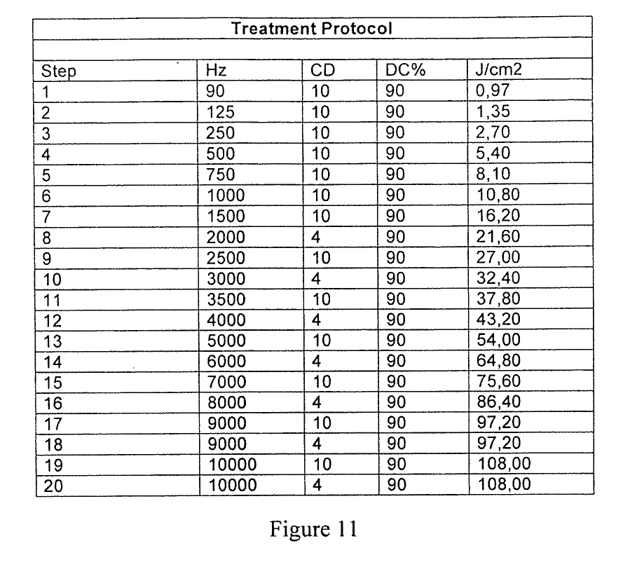

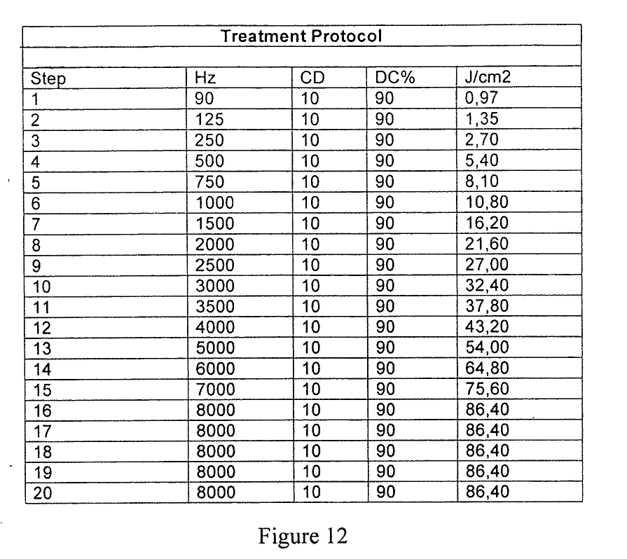

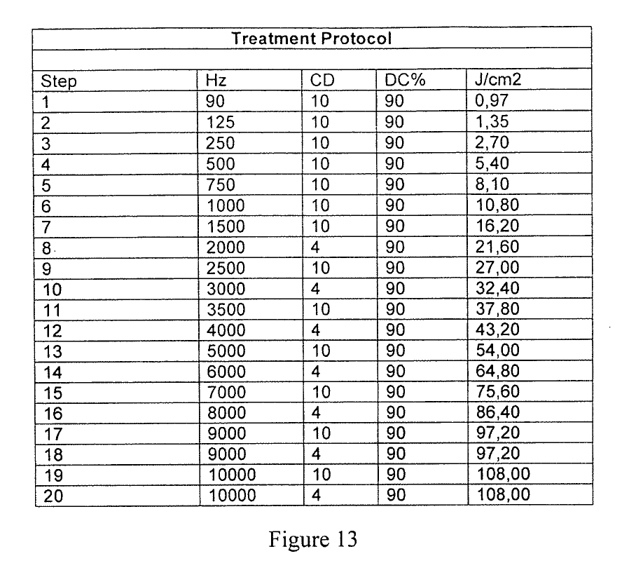

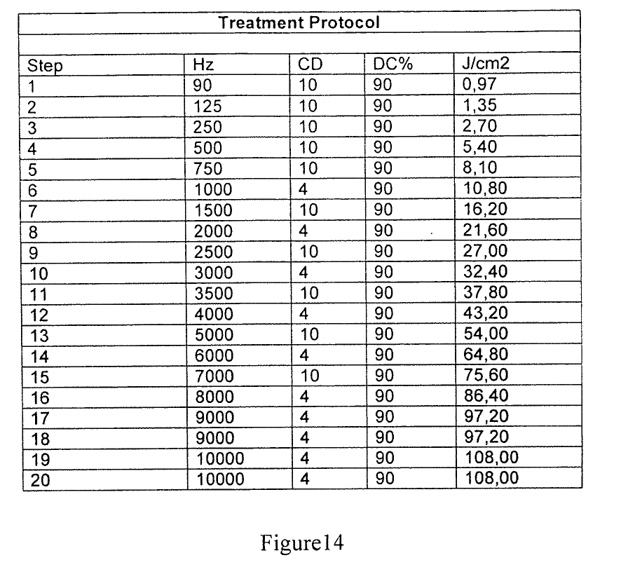

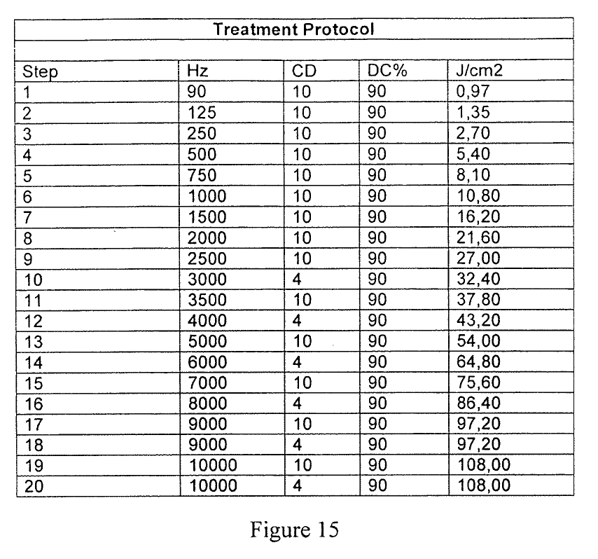

After the treatment is determined, the clinician accesses the low level laser therapy apparatus 10 to select an associated laser treatment protocol (forming the laser component) for the diagnosed injury from a menu of available pre-stored treatment protocols (step 312). Examples of the characteristics of various treatment protocols may be seen in FIGS. 8 to 15. In the preferred embodiment, the laser component comprises a plurality of laser treatments (preferably five (5) minutes in length) with the exercise rehabilitation components scheduled between each of the plurality of laser treatments.

Once the treatment protocol is selected, the clinician applies the laser treatment to the injured site (step 314) for the allotted five minute interval. One example of how the low level laser therapy may be administered is described above with respect to FIGS. 5 and 6. After the five-minute treatment has been completed, the clinician performs a manual component (step 316) followed by the patient performing the exercise rehabilitation component (step 318) which is completed by the patient encompassing the treatment program. After the patient has completed the exercise rehabilitation component, the patient returns to the clinician for the next of the plurality of low level laser treatments. The clinician then determines if the individual (patient) has received all of their laser treatments according to the described protocol (step 320). If the patient has completed the laser treatment portion of the protocol, the treatment protocol is deemed completed (step 322).

If the patient is scheduled to receive more laser treatments, the patient returns for a next of the plurality of the five minute laser treatments (step 314). This pattern of low level laser treatment and exercise rehabilitation therapy is repeated until the patient has completed all of the laser treatments in the treatment protocol. There are preferably 15 to 20 levels of laser treatments for each protocol but it will be understood that other numbers of levels are contemplated.

In a more detailed example, for example after a clinician has diagnosed an individual with a Cervical: Acute Pain/Radiculitis musculo-skeletal condition, the clinician activates the laser therapy apparatus 10 to select a Cervical: Acute Pain/Radiculitis treatment protocol (such as shown in FIG. 8). In this treatment program, the low level laser treatment comprises fifteen (15) laser treatments.

In operation, after the injury has been diagnosed and the Cervical: Acute Pain/Radiculitis treatment protocol determined (step 310) and selected (step 312), the patient proceeds to undergo treatment from the clinician, or therapist. As discussed above, the low level laser therapy apparatus 10 is activated and used to preferably emit low level laser therapy to the injured area. It will be understood that any laser device which is capable of providing the parameters as listed in the treatment protocols of FIGS. 8 to 15 may be used. It will be understood that other treatment protocols to treat other musculo-skeletal conditions are contemplated with only slight variations in the parameters of the treatment levels.

In the initial treatment, the clinician sets the laser therapy device to emit low laser energy (step 314) to the injured area having a frequency of 90 Hz, a cadence of 10, a duty cycle of 90% and an energy level of 0.97 J/cm². This low level laser therapy is preferably applied for 5 minutes. After the laser treatment is completed, the patient is asked to perform predetermined exercise rehabilitation components (step 318) prior to the next laser treatment which preferably occurs no less that 24 hours after the pervious treatment. After the patient performs the exercise rehabilitation component(s), the patient returns for the next laser treatment session, whereby low level laser therapy having a frequency of 125 Hz, a cadence of 10, a duty cycle of 90% and an energy of 1.35 J/cm² is applied for five minutes by a low level laser therapy apparatus, such as shown in 10. After completing the second session of low level laser therapy (step 314), the patient performs further exercise rehabilitation components (step 318) prior to the third session of treatment. As discussed above, there is preferably at least 24 hours between each of the laser treatment sessions. After the patient performs the exercise rehabilitation components, the patient returns for the third session of laser treatment, which in this embodiment has a frequency of 125 Hz, a cadence of 10, a duty cycle of 90% and an energy of 1.35 J/cm². Once again, this treatment is preferably applied for 5 minutes. The patient performs the exercise rehabilitation components before returning for the fourth laser treatment session. This process continues until the patient has completed each of their laser treatment sessions (which in this embodiment is 15) (step 320) after which the treatment protocol program is deemed complete (step 322). It will be understood that the listed treatment protocol is the one which the patient is expected to undergo, however, the treatment is subject to ongoing diagnoses by the clinician (after each treatment level) whereby the patient may be required to repeat the treatment at a previous level or a decreased setting.

Depending on the nature of the treatment program, the exercise rehabilitation components of the program may not change between laser treatments or they may be different from each other. The manual and exercise rehabilitation components of the program are meant to supplement the laser treatments.

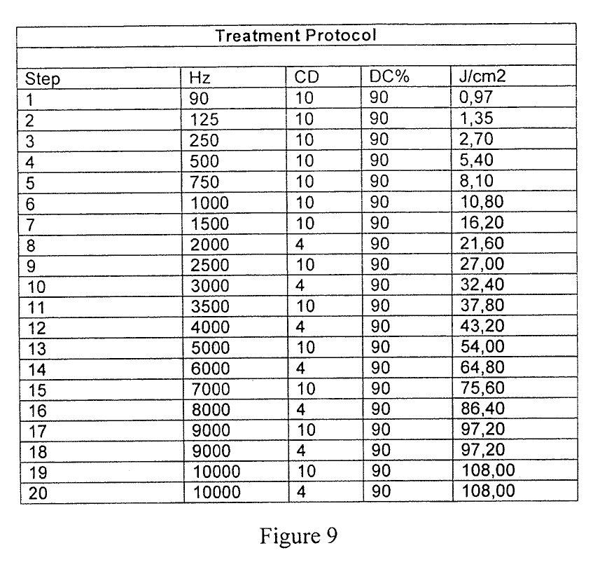

For other treatment protocols, the properties of the laser treatments are listed in FIGS. 9 to 15. As will be understood, with the protocol disclosed above, the laser treatment alone is quite beneficial in the treatment of various muscle injuries but when combined with a set of exercise rehabilitation components provides the patient with an enhanced recovery program and improved musculo-skeletal conditions.

It will be understood that the properties of the laser treatments in the above-identified treatment protocol programs are the preferred embodiments but as one skilled in the art will understand, these values may be altered without affecting the overall laser therapy being administered to an individual.

In an alternative embodiment, the invention may also be used on animals as a diagnostic tool or a therapy tool.

In yet another embodiment, the apparatus may be portable in order to allow physicians to carry the apparatus for treatment on the spot. In this embodiment, the control device is powered by the battery 64 and has at least one probe. As discussed above, the probe may include the CPU, the set of treatment control buttons and a battery and may be used as a stand-alone low-level laser therapy unit.

It will further be understood that although only five diodes are shown in the multi-cluster probe, the probe may include any number of diodes with the only restriction being the size of the probe head.

For each step of a treatment program, i.e. consecutive treatments, there is an increase of frequency and as a result an increase in the amount of photon energy in units of Joules/cm². In order to assist the user, the information displayed on the display window is replicated in the display window of the probe so that the user has immediate access to such information and does not have to return to the control device to see the parameters of the treatment.

Examples of various injuries which may be treated with the apparatus and treatment protocols of the present invention include: Cervical Spine injuries, Thoracic/Lumbar injuries, Sacro Iliac injuries, Sports Injuries, Peripheral Muscle injuries, Carpal Tunnel Syndrome or Plantar Fascitis. Other injuries which the treatment protocols may be geared towards healing include Osteoarthritis, Chronic Low Back Pain Acute Sports Injury—Strain/Sprain Soft Tissue Injury—Peripheral Joints, Acute Soft Tissue Injury of the Cervical, Thoracic and Lumbar Spines, Chronic Soft Tissue Injury—Cervical Spine, Chronic Soft Tissue Injury—RSI, Carpal Tunnel Syndrome, Fibrous/Scar Tissue and Tension/Stress Headaches.

In various embodiments, the present invention incorporates a series of various treatment protocols which include the use of a duty cycle and/or an apparatus with deviation in the delivery of the low level laser energy of less than +/−5%, to remove the necessity of manual adjustment, thereby reducing the complexity and significant training of qualified personnel for its administration.

For those embodiments where the probe includes a CPU, the probe CPU also provides constant impendence level checks on the amount of power being supplied by the diodes during treatment. These checks are performed in order to constantly monitor the impedance levels to ensure that the level of treatment being applied remains within required tolerance levels. If required, extra voltage and current is supplied to the laser diodes in order to compensate for any issues with the level of laser treatment being supplied to the patient. Furthermore, this reduces the need for instrument calibration since the processor within the probe is constantly verifying the required power levels of the diodes.

The above-described embodiments of the present invention are intended to be examples only. Alterations, modifications and variations may be effected to the particular embodiments by those of skill in the art without departing from the scope of the invention.

What is claimed is:

1. Apparatus for delivering low level laser therapy comprising:
at least one probe for delivering said low level laser therapy;
a processor, connected to said at least one probe, for transmitting signals to said probe corresponding to said low level laser therapy;
wherein, after receiving said signals, said low level laser therapy is delivered by said probe at a wavelength of between 600 and 1100 nm.

2. The apparatus of claim 1 wherein said processor is housed in a control device.

3. The apparatus of claim 1 wherein said probe is connected to said control device via a probe connector.

4. The apparatus of claim 1 wherein said apparatus comprises one probe.

5. The apparatus of claim 4 wherein said processor is housed in said one probe.

6. The apparatus of claims 1 to 5 wherein said apparatus further comprises means for adjusting said low level laser therapy values.

7. The apparatus of claim 6 wherein said means include at least one of:
means for varying a cadence of said low level laser therapy;
means for varying a frequency of said low level laser therapy;
means for varying an energy level of said low level laser therapy; and
means for varying a duty cycle of said low level laser therapy.

8. The apparatus of claims 1 to 3 wherein said at least one probe comprises:
at least one diode for delivering said low level laser therapy.

9. The apparatus of claim 8 wherein said at least one probe comprises 5 diodes.

10. The apparatus of claim 1 wherein said low level laser therapy is delivered at a wavelength of 905 nm.

11. The apparatus of claims 8 or 9 wherein said diode is spring-loaded.

12. The apparatus of claim 1 wherein said at least one probe comprises:
means for testing said probe to determine if said probe is functional.

13. The apparatus of claim 12 wherein said means for testing comprises:
means for measuring an amount of photon energy being delivered by the at least one probe; and
means for comparing said measured amount with an expected value.

14. The apparatus of claim 13 further comprising means for powering down said probe if said measured amount of photon energy does not equal said expected value within an allowable tolerance.
